(19) United States
(12) Patent Application Publication
(10) Pub. No.: US 2002/0090642 A1
(43) Pub. Date: Jul. 11, 2002

(54) METHODS FOR DETERMINING THE PROGNOSIS OF BREAST CANCER USING ANTIBODIES SPECIFIC FOR THYMIDYLATE SYNTHASE

(76) Inventors: PATRICK G. JOHNSTON, BETHESDA, MD (US); CARMEN J. ALLEGRA, NORTH POTOMAC, MD (US)

Correspondence Address: WILLIAM B KEZER, TOWNSEND AND TOWNSEND AND CREW, TWO EMBARCADERO CENTER, 8TH FLOOR, SAN FRANCISCO, CA, 941113834

(21) Appl. No.: 09/152,647

(22) Filed: Sep. 14, 1998

Related U.S. Application Data
(63) Continuation of application No. 08/762,264, filed on Dec. 9, 1996, now abandoned.
(60) Provisional application No. 60/008,494, filed on Dec. 11, 1995.

(51) Int. Cl. G01N 33/53; G01N 33/574; G01N 33/537; G01N 33/543

(52) U.S. Cl. 435/7.1; 435/7.23; 435/7.5; 435/7.92

(57) ABSTRACT

Methods for determining the prognosis of a patient afflicted with breast cancer, comprising;

(a) obtaining a solid breast tumor tissue sample from the patient;

(b) measuring the level of thymidylate synthase expression in the tissue sample; and

(c) comparing the level of thymidylate synthase expression with thymidylate synthase expression levels from a group of standard breast cancer tissue samples, wherein each of the standards has a known thymidylate synthase expression level, a known disease-free survival rate and known overall survival rates, to determine the prognosis of the patient.

### BACKGROUND OF THE INVENTION

Thymidylate synthase (TS; EC.2.1.1.45) provides the sole de novo source of thymidylate for DNA synthesis. It is also a critical therapeutic target for the fluoropyrimidine cytotoxic drugs, such as fluorouracil (5-FU) and fluorodeoxyuridine (FUdR). In both clinical and preclinical studies increased expression of TS protein has been associated with resistance to 5-FU (see, Johnston, et al., Cancer Res. 52:4306-4312 (1992)).

The quantitation of TS has traditionally been performed using enzymatic biochemical assays; however these assays have major limitations when applied to human tumor tissue samples. Recently, monoclonal antibodies have been developed to human TS that have the required sensitivity and specificity to detect and quantitate TS enzyme in formalin-fixed tissue sections (see, Johnston, et al., Cancer Res. 51:6668-6676 (1991)).

In 1990, an NIH Consensus Conference on treatment of early-stage breast cancer made recommendations for future research. The first proposed direction of future research was “to refine existing prognostic factors, by . . . (d) developing and using new and existing tissue and clinical data banks for the study of prognostic factors.” See, J. Am. Med. Assn. 265:391-395 (1991). Since that time, a host of prognostic factors have been studied, and several editorials have provided guidelines how such research should be carried out and integrated with existing knowledge (see, McGuire, J. Natl. Cancer Inst. 83:154-155 (1991) and Osborne, J. Clin. Oncol. 10:679-682 (1992)). In particular, it was pointed out that prognostic factors should be tested on data sets large enough to permit meaningful statistical analysis, that they be compared with established prognostic factors, and that a factor should be analyzed not only in terms of its prognostic significance but also in terms of its predictive significance, i.e. whether it can predict the value of a particular adjuvant treatment. Accordingly, the clinical importance of TS enzyme expression has now been examined in 488 patients with early stage breast cancer. The results have led to new methods for determining the prognosis of a patient afflicted with breast cancer and also for predicting the benefit of chemotherapy for that patient.

### SUMMARY OF THE INVENTION

The present invention provides a method for determining the prognosis of a patient afflicted with breast cancer, comprising;

(a) obtaining a solid breast tumor tissue sample from the patient;

(b) measuring the level of thymidylate synthase expression in the tissue sample; and

(c) comparing the level of thymidylate synthase expression with thymidylate synthase expression levels from a group of standard breast cancer tissue samples, wherein each of the standards has a known thymidylate synthase expression level, a known disease-free survival rate and known overall survival rates, to determine the prognosis of the patient.

The present invention further provides a method for predicting the benefit of chemotherapy for a patient afflicted with breast cancer, comprising;

(a) obtaining a solid breast tumor tissue sample from the patient;

(b) measuring the level of thymidylate synthase expression in the tissue sample; and

(c) comparing the thymidylate synthase expression level with thymidylate synthase expression levels from a group of standard breast cancer tissue samples, in which each of the standards has a known thymidylate synthase expression level, known courses of chemotherapeutic treatment, known disease-free survival rates and known overall survival rates, to predict the benefit of chemotherapy the patient.

In each of these methods, thymidylate synthase expression levels are preferably measured with an antibody specific for thymidylate synthase.

### BRIEF DESCRIPTION OF THE DRAWINGS

FIG. 1 is a graph which illustrates the disease-free survival according to TS expression levels with node-positive patients.

FIG. 2 is a graph which illustrates the disease-free survival according to treatment with node-positive patients having high TS.

FIG. 3 is a graph which illustrates the overall survival according to TS expression levels with node-positive patients.

FIG. 4 is a graph which illustrates the overall survival according to treatment with node-positive patients having high TS.

### DETAILED DESCRIPTION OF THE INVENTION

Abbreviations and Definitions

Abbreviations used herein have the following meanings: TS, thymidylate synthase; CMF, cyclophosphamide-methotrexate-fluorouracil; ER, estrogen receptor; PR, progesterone receptor; PBS, phosphate-buffered saline; BSA, bovine serum albumin; DFS, disease-free survival; OS, overall survival; 5-FU, 5-fluorouracil.

Description of the Embodiments

The present invention derives from the surprising discovery that high TS expression is associated with a poor prognosis in node-positive, but not in node-negative breast cancer patients. This discovery was made upon examination of tissue samples for a clinical trial involving 2504 patients, the details of which have been provided elsewhere (see, N. Engl. J. Med. 319:677-683 (1988) and N. Engl. J. Med. 320:491-496 (1989)). Additionally, thymidylate synthase expression was not found to be correlated with other prognostic factors including tumor size, ER status, PR status, tumor grade, vessel invasion, and histology. In terms of treatment, the results presented herein further support the conclusion that adjuvant treatment in node-positive patients is more effective in the high TS group than in the low TS group.

As demonstrated below in the Examples, thymidylate synthase protein expression in node-positive breast cancer patients predicts clinical outcome. Several reasons may account for this finding. First, TS expression is a marker of proliferation since it plays a key role in DNA nucleoside precursor synthesis. It has recently been shown that TS expression is high in proliferating cells and approximately 20-fold lower in resting cells (see, Pestalozzi, Brit. J. Cancer 00:000-000 (1995). Accordingly, TS expression was compared with the expression of the proliferation marker Ki-67 and evaluated by staining on paraffin-embedded tissue with the MIB-antibody in 202 node-negative and 264 node-positive samples (data not shown). No correlation between Ki-67 and TS was found. Thus, it is unlikely that the prognostic significance of TS expression is solely due to its correlation with proliferative activity. A second reason to account for the prognostic significance of TS expression comes from laboratory studies demonstrating that TS protein binds to c-myc mRNA to form a ribonucleoprotein complex (see, Chu, et al., Mol. Cell. Biol. 14:207-213 (1994). It is thus possible that TS protein is involved in regulatory and/or other cellular processes related to tumor growth.

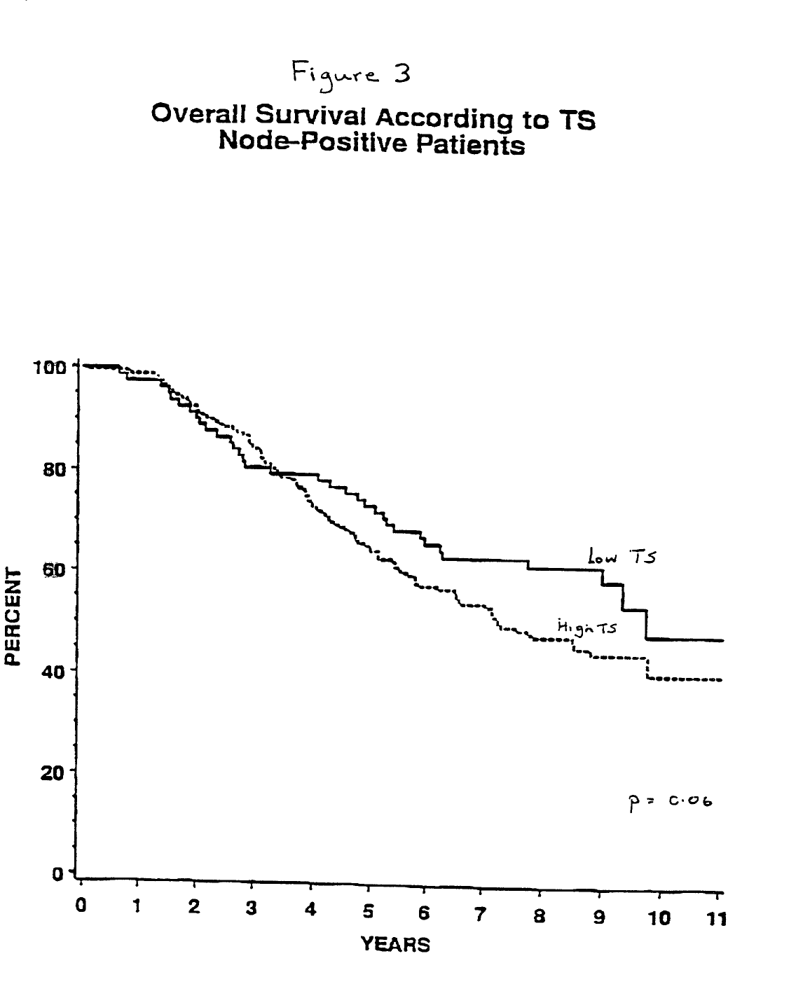

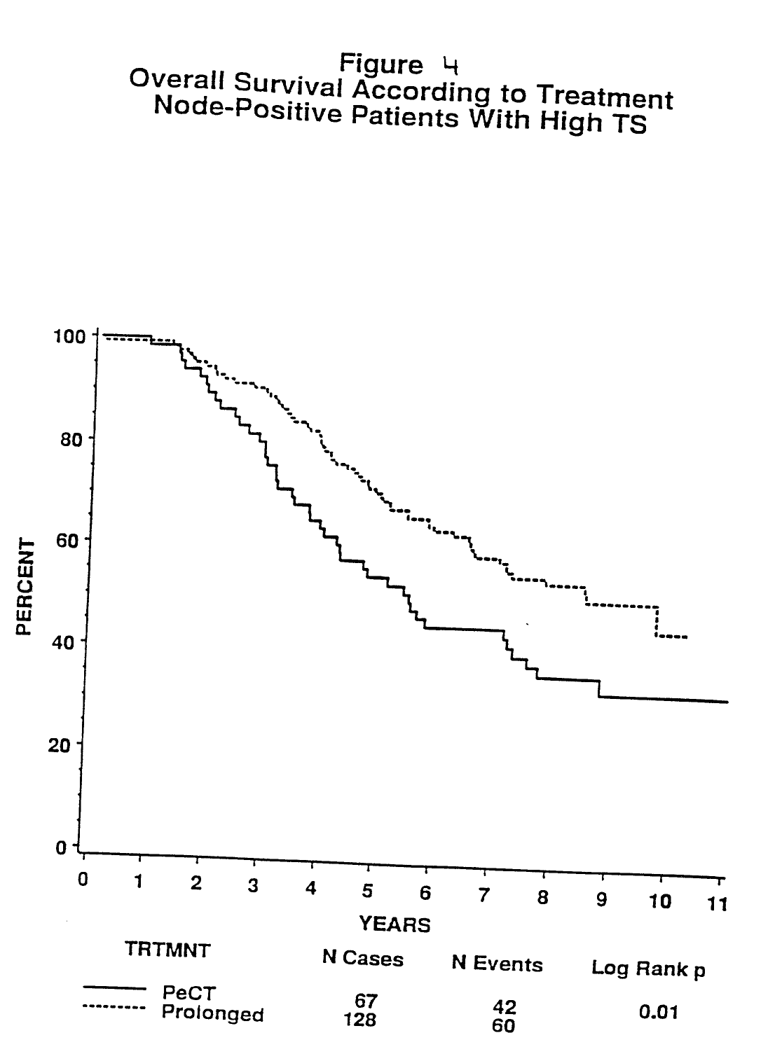

In addition to its role in DNA nucleoside precursor synthesis, TS represents an important therapeutic target for 5-fluorouracil (5-FU), which is part of the adjuvant CMF regimens used in this trial. The CMF regimens comprise a treatment with cyclophosphamide, methotrexate and the above-noted fluorouracil. Accordingly, TS expression can be compared with the treatment effect of a 5-FU containing regimen. In node-negative patients there was no correlation of disease-free survival (DFS) nor of overall survival (OS) according to treatment in patients with high TS as well as in patients with low TS. On the contrary, in node-positive patients there was a correlation of DFS and OS according to treatment in patients with high TS (see Examples below and FIGS. 3 and 4, p=0.06), but not in patients with low TS. Although this apparent benefit of chemotherapy in patients with high TS levels but not low TS levels has been observed previously in a rectal cancer trial of adjuvant therapy (NSABP R-01, Johnston, et al., J. Clin. Oncol. 12:2640-2647 (1994)), it is not easily explained. In cell lines and experimental tumors, high TS levels are associated with fluoropyrimidine resistance. Likewise, in gastric and colorectal cancer high TS RNA-levels and protein levels are associated with lack of responsiveness to fluoropyrimidine-based chemotherapy (see, Johnston, et al., Am. Soc. Clin. Oncol. 13:196 (abstract) (1994)).

In conclusion, the level of TS expression predicts for DFS and OS in node-positive, but not node-negative breast cancer patients. The results provided herein also support the conclusion that node-positive breast cancer patients with high TS levels can derive the greatest benefit from adjuvant chemotherapy.

In view of the above surprising discoveries, the present invention provides a method for determining the prognosis of a patient afflicted with breast cancer, comprising;

(a) obtaining a solid breast tumor tissue sample from the patient;

(b) measuring the level of thymidylate synthase expression in the tissue sample; and

(c) comparing the level of thymidylate synthase expression with a group of standard breast cancer tissue samples, wherein each of the standards has a known thymidylate synthase expression level, a known disease-free survival rate and known overall survival rates, to determine the prognosis of the patient.

In this method, a solid breast tumor tissue sample is obtained from a patient, preferably a node-positive patient. As used herein, the terms “node-positive” and “node-negative” refers to patients whose breast cancer has and has not metastasized to the lymph nodes, respectively. Typically, the tissue sample is obtained by conventional techniques known to those of skill in the art for obtaining biopsy samples.

The tissue which is isolated can be used directly or it can be embedded in, for example, paraffin and stored for future use. The term “embedded” refers to a sample that has been infiltrated with a material to provide mechanical support and thereby reduce sample deformation during processes such as sectioning (preparing thin slices for viewing using a microscope). Embedding materials include waxes, such as paraffin wax, epoxies, gelatin, methacrylate, nitrocellulose, various polymers and the like. The term “non-embedded” refers to a sample that is not embedded, and was not previously embedded. When a tissue sample is embedded in, for example, paraffin, for future use, it will preferably be in sections of about 6 micron thickness. Upon removal from storage, the paraffin-embedded tissue sections will be deparaffinized using a relatively non-polar aprotic organic solvent such as xylene, and then rehydrated using graded alcohols followed by phosphate-buffered saline (PBS). Other suitable solvents for removing the embedding support include aliphatic or aromatic hydrocarbon solvents such as toluene, heptanes, octanes, benzene, acetone and acetonitrile.

Following isolation of the tissue sample, the thymidylate synthase expression levels are measured in the sample. A number of methods known to those of skill in the art can be used for measuring TS in a sample. For example, protein levels can be determined by substrate interactions using either a labeled or unlabeled substrate, or by contacting the tissue sample with an antibody specific for thymidylate synthase. Preferably, the TS levels are determined by contacting the sample with an antibody which is specific for thymidylate synthase (TS). Antibodies which are specific for TS have been described. See, Johnston, et al., Cancer Res. 51:6668-6676 (1991) and Johnston, et al., Cancer Res. 52:4306-4312 (1992). Briefly, hybridomas, for example, murine hybridomas, that produce monoclonal antibodies that are immunoreactive with the enzyme thymidylate synthase can be prepared and selected for as described in the Examples that follow. For example, mice (i.e. balb/c mice) can be immunized with the thymidylate protein by intraperitoneal injection. After sufficient time has passed to allow for an immune response, the mice can be sacrificed and the spleen cells obtained and fused, advantageously, with myeloma cells, using techniques well known in the art. The resulting fused cells, hybridomas, are then grown in a selective medium, and the surviving cells grown in such medium using limiting dilution conditions. After cloning and recloning, hybridomas can be isolated that secrete antibodies (for example, of the IgG or IgM class) directed to the target protein, thymidylate synthase, which has a molecular weight of 36,000 daltons.

Hybridomas that secrete antibodies used in the present invention have been deposited with the American Type Culture Collection, and have been assigned the ATCC number ______.

Additionally, fragments of the TS-specific monoclonal antibodies, such as Fab or F(ab¹)₂ can also be used in the present invention. The antibody fragments can be obtained by conventional techniques, for example, by digestion of the antibody using papain or pepsin.

The above-specified examples of the monoclonal antibodies of the present invention are of the IgG and IgM classes, and are obtained from a murine source, however, this is not meant to be a limitation. The antibodies of this invention and those functionally equivalent thereto (that is, capable of specific binding to the above-described TS antigens) are within the scope of the invention, whether from a murine source, or other mammal, including human, or combinations thereof. Likewise, use of antibodies of other classes such as IgA, IgE, etc., and isotypes within the classes, is also within the intended scope of the invention.

Isolation and purification of the monoclonal antibodies can be accomplished using various conventional methods, which free monoclonal antibodies from other proteins and contaminants (see, for example, Goding, in MONOCLONAL ANTIBODIES: PRINCIPALS AND PRACTICE, Chapter 4, 1986). Preferably, the monoclonal antibodies used in the present invention are able to detect TS in fresh frozen human tumor tissue and paraffin-embedded tissues. More preferably, the antibody used is an antibody such as those described in Johnston, et al., Cancer Res. 52:4306-4312 (1992).

The methods of contacting antibodies with the tissue sample and determining levels of thymidylate synthase expression can be carried out using standard procedures, including immunoassays which qualitatively and quantitatively analyze the target protein. A general overview of the applicable technology can be found in Harlow and Lane, Antibodies: A Laboratory Manual, Cold Spring Harbor Pubs., N.Y. (1988). Either monoclonal or polyclonal antibodies specific for the target protein can be used in various immunoassays. Such assays include ELISA, competitive immunoassays, radioimmunoassays, western blots, indirect immunofluorescent assays and the like. In a particularly preferred embodiment, an immunohistochemical technique is used with an avidin-biotin complex.

Once the expression levels of TS have been determined in the patient of interest, the levels can be compared with the expression levels from a study cohort, such as that described in Example 2, below, and projections can be made as to the patient's prognosis.

The importance of the TS enzyme level as a mechanism of drug resistance is indicated by studies demonstrating that acute induction of TS protein as well as stable amplification of TS may be associated with 5-FU resistance in human breast and colon cancer cell lines (Spears, et al., Cancer Res. 42:450-456 (1982); Washtein, Mol. Pharmacol. 25:171-177 (1984); Chu, et al., Cancer Res. 50:5834-40 (1990); Keyomarsi, et al., J. Biol. Chem. 263:14402-14409 (1988); Scanlon, et al., Proc. Natl. Acad. Sci. USA 85:650-653 (1988); Bradford, Anal. Biochem. 72:248-254 (1976); and Hsu, et al., J. Histochem. Cytochem. 29:577-585 (1981)). The clinical relevance of acute TS indication has been suggested by an in vivo study carried out on tumor biopsy samples obtained from patients with breast carcinoma that documented a 2- to 6-fold increase in TS 24 hours post-fluorouracil therapy swain (see, Lippman, et al., J. Clin. Oncol. 7:890-896 (1989)). These in vivo and in vitro studies suggest that the ability of a tumor to overexpress TS in response to cytotoxic agents is important in the clinical development of tumor resistance.

Until now the measurement of TS levels in human tissue has been carried out by using the radiolabeled FdUMP binding assay (Lockshin, et al., Biochem. Pharmacol. 30:247-257 (1981) and Lockshin, et al., J. Biol. Chem. 254:12285-12288 (1979)). This assay is performed on a cytosolic extract wherein relatively large quantities of tissue are required and the cellular specificity is lost. The biochemical study assay cannot discriminate between areas of the tumor with differing morphologies, nor can it measure TS on a cell-to-cell basis, and, as tissues and cell preparations are a composite of a heterogenous population, any measurement of TS enzyme is confounded by the degree of contamination by cells other than those of interest. In comparison to the biochemical assay, the availability of a quantitative immunohistochemical assay to measure TS in human cells and tissues is advantageous in the examination of cells in tissue sections. Such an assay allows TS measurement in primary and metastatic tumor samples on a cell-by-cell basis, and facilitates detailed correlations between the level of TS and various clinical and morphological parameters. This information can be of value in patient selection for 5-FU treatment. In addition to TS quantitation in tissues and cells, the antibodies used in the present invention will permit accurate studies of TS production and its regulation by drugs such as 5-FU and methotrexate and biologicals such as the interferons, both in vitro and in vivo. The ability of antibodies to recognize and distinguish native and complexed protein will help provide information about stability of the TS-FdUMP-folate complex and its modulation by exogenous folates.

The invention also relates to a diagnostic kit for detecting the presence of thymidylate synthase, which, in one embodiment, comprises in combination:

an insoluble surface or support, preferably containing microtiter wells;

thymidylate synthase or antigenic fragments thereof which are bound to the support;

at least one monoclonal antibody as described above, or a binding fragment thereof, which specifically binds to the antigenic portions of thymidylate synthase; and

means for detecting the amount of antibodies in a test which bind to the monoclonal antibody or binding fragments.

Still further, the present invention provides a method for predicting the benefit of chemotherapy for a patient afflicted with breast cancer, comprising;

(a) obtaining a solid breast tumor tissue sample from the patient;

(b) measuring the level of thymidylate synthase expression in the tissue sample; and

(c) comparing the thymidylate synthase expression level with a group of standard breast cancer tissue samples, in which each of the standards has a known thymidylate synthase expression level, known courses of chemotherapeutic treatment, known disease-free survival rates and known overall survival rates, to predict the benefit of chemotherapy the patient.

The patients, methods of obtaining tissue samples and measuring the level of TS expression, are as described above. Once the level of TS expression has been determined, the level can be compared with levels from a study cohort in which members of the study have been subjected to various therapies and for which survival rates are known (see Example 2).

The following examples are offered solely for the purposes of illustration, and are intended neither to limit nor to define the invention.

### EXAMPLES

### Example 1

This example illustrates the development and selection of hybridomas which produce monoclonal antibodies specific for thymidylate synthase.

Recombinant thymidylate synthase protein was a kind gift from Dr. D. Santi (University of California). Polyethylene glycol was purchased from J. T. Baker. Pristan was purchased from Aldrich Chemical Co. (Milwaukee, Wis., USA). Peroxidase labeled affinity purified goat antimouse immunoglobulins were purchased from (Kirkegaard and Perry Laboratories, Md., USA). [6-³H] 5 Fdump (18 ci/mmol) was purchased from Moravek Biochemicals (Brea, Calif., USA). ¹⁴C-Methionine was purchased from NEN (Boston, Mass., USA). 96 well Immulon Plates were purchased from Dynatech (Chantilly, Va., USA). A.B.C. Immunoperoxidase kits were purchased from Vector Laboratories (Burlingame, Calif., USA). All other chemicals were obtained from Sigma or NIH supply.

1.1 Immunization Fusion and Cloning

Five female balb/c mice (10 weeks old) were injected intraperitoneally with recombinant human thymidylate synthase (r TS) 10 μg per mouse. The r TS peptide had been emulsified in Freund's complete adjuvant. The mice were subsequently boosted twice with r TS 10 μg/mouse at 21 day intervals. A mouse with high reactivity (≧1/100,000 by ELISA) was chosen and injected with r TS 10 μg in 0.5 mL phosphate buffer saline (PBS) 4 days prior to fusion. Spleen cells (1×10⁸) from the immunized mouse were fused with 2×10⁷ P3×63 Ag 8 variant 653 myeloma cells by using 50% (v/v) polyethylene glycol 3350 (J. T. Baker Chemical Co.) as a fusing agent. The fusion procedure of Galfre and Milstein was used (Galfre et al., Nature 266:550 (1977)). The fused cells were plated onto 96 well plates and screened for monoclonal antibody production by ELISA starting on day 28 post fusion. The ELISA-positive hybridomas were cloned and recloned three times at 1, 10 and 50 cells per 96-well plates on feeder cells. The resultant stable colonies were expanded into 25 CM₂ Falcon Flasks. In this fusion 412 hybridomas resulted from plating of fused cells into 768 wells (53%) ELISA positive clones were detected in 114/412 (27%) wells and 38 of these were positive by subsequent western blot assays. The ELISA positive hybridomas were cloned and recloned by limiting dilution techniques. Seven stable hybridomas were produced which secreted antibodies to thymidylate synthesis. These were identified as TS 102, TS 105, TS 106, TS 109, TS 110, TS 111A and TS 111B.

1.2 Preparation and Purification of Ascitic Fluid

The mice were injected intraperitoneally with 0.5 mL of Pristan (Aldrich Chemical Co.) and 10-14 days later inoculated with 1×10⁶ hybridoma cells per mouse. The ascitic fluid was collected at 2-3 day intervals until the animal was sacrificed. The ascitic fluid was purified by precipitation with 40% ammonium sulphate and high pressure liquid chromatography (Liang, et al., Biochem. Biophys. Res. Commun. 128:171-178 (1985)). Isotypic analysis using immunodiffusion techniques indicated that 4 cell lines produced antibodies of the IgG class, and 1 cell line produced antibodies of the IgM class. The class and subclass of the monoclonal immunoglobulins produced were determined by Ouchterlony analysis with antisera specific for μ chains and for IgG and IgM subclasses (Ouchterlony, in HANDBOOK OF EXPERIMENTAL IMMUNOLOGY, Weir D. M. (ed.), Oxford and Edinburgh: Blackwell 1976, pp. 655-707). Antibodies TS 106, TS 109, TS 110 and TS 111A belonged to the IgG1 class, while antibody TS 111B belonged to the IgM class.

1.3 Iodination of Recombinant Human TS Protein

¹²⁵I-rhTS was prepared using the purified rhTS protein (EC 2.1.1.45). The soluble lactoperoxidase technique was used to label the rhTS with ¹²⁵I (FLPC for Monoclonal Antibody Purification (Pharmacia brochure)). The labeled protein was separated from unreacted Na ¹²⁵I by passing the iodination mixture through a SEPHADEX® G-25 column, equilibrated and eluted with a buffer containing 0.01 M PBS, pH 7.4, 0.15 M NaCl and 0.1% BSA.

1.4 ELISA

An Immulon II 96-well plate was coated with 50 μL of thymidylate synthase (1 μg/mL) in coating buffer (0.015 M NaHCO₃) overnight at 4° C. The wells were then washed thoroughly with PBS/TWEEN® (1 N PBS, 0.1% Azide 0.05% TWEEN®) and incubated with 100 μL of bovine serum albumen 5 mg/mL at room temperature for 1 hour. After washing three times with PBS/TWEEN®, 50 μL of hybridoma supernatant was added and incubated at room temperature for 2 hours. The unbound antibody was then washed off with three further washes of PBS/TWEEN® and 50 μL of alkaline phosphatase conjugated antimouse antibodies were added for 4 hours. The wells were then washed three times in PBS/TWEEN® and 50 μL of substrate solution containing (p-nitrophenyl phosphate (PNP) 40 mg in 20 mL of 100 mM NaHCO₃ and 10 mg MgCl₂, pH 8.0) was added to each well. The substrate gave a greenish yellow color and the optical density of the reaction was assessed at 405 nm in a microelisa autoreader (Dynatech). Determination of positive hybridomas cultures was based on signals >0.5 units above background.

### Example 2

This example illustrates the correlation between disease-free survival and overall survival for node-positive and node-negative breast cancer patients having high and low thymidylate synthase levels.

2.1 Trial Design

A total of 2504 patients were entered onto the International Breast Cancer Study Group (Ludwig) Trial V between November 1981 and December 1985. Node-negative breast cancer patients (N=1275) were randomized between a single cycle of perioperative chemotherapy (PeCT) and no adjuvant therapy (NoCT). For the single cycle perioperative chemotherapy, patients were treated with one combined dosage of cyclophosphamide, methotrexate and fluorouracil, immediately following breast and tumor removal. Node-positive patients (N=1229) were assigned to one of three treatments: perioperative chemotherapy (administered as with the node-negative patients), conventionally-timed chemotherapy (administered over a prolonged time period) or both. The perioperative chemotherapy regimen was also a combination of cyclophosphamide, methotrexate and fluorouracil. The conventionally-timed chemotherapy regimen was the same combination plus prednisone. The trial and the clinical results have been described in detail elsewhere (see, N. Engl. J. Med. 319:677-683 (1988) and N. Engl. J. Med. 320:491-496 (1989)). In this report, the two treatment arms which received conventionally timed chemotherapy (with or without perioperative chemotherapy) have been combined because the prognosis in these two groups is very similar.

2.2 Study Cohort

The TS study samples consisted of tissue sections from paraffin-embedded blocks available from 555 patients. The clinical and pathologic data available for all patients were kept separately in a computerized data bank at the Division of Biostatistics of the Dana-Farber Cancer Institute in Boston, Mass. Median follow-up at the time of this analysis is 10 years. On 67 of 555 sections no invasive tumor was visible histologically, and these were excluded. Thus, 488 cases were available for statistical analysis. The results below are from 210 node-negative and 278 node-positive cases.

2.3 Immunohistochemical Labeling

Paraffin-embedded tissue sections 6-μM thick were deparaffinized in xylene, rehydrated through graded alcohols, and washed in phosphate-buffered saline (PBS). The TS 106 monoclonal antibody was applied using the avidin-biotin complex (ABC) immunohistochemical technique (see, Hsu, et al., Am. J. Clin. Pathol. 75:734-738 (1981)).

2.4 Tissue Evaluation

The slides were examined and scored independently by two observers blinded to both the clinical and pathologic data. TS expression was quantitated using a visual grading system based on the intensity of staining (0-3+) as well as its extent, focal (<25% of tumor staining positive) or diffuse (>25% of tumor staining positive). Intensity levels 0 to 1 were grouped together and considered low-intensity, whereas 2 to 3 staining intensity was considered high-intensity staining. There was close agreement (>85%) in the TS evaluation between both investigators. In those cases of disagreement, final grading was determined by consensus. Duplicate samples were included in the study as internal controls for TS immunostaining and interpretation. The TS staining intensity was similar from one immunohistochemical experiment to the next and the interpretation had 100% concordance.

2.5 Statistical Analysis

Estimates of 5-year and 10-year disease-free survival (DFS) and overall survival (OS) and their standard errors were calculated by the Kaplan-Meier method and Greenwood's formula, respectively. Logrank tests (reported on the figures) are not adjusted for other prognostic factors. Hazard ratios were estimated from Cox proportional hazards regression models, with no adjustments for other prognostic factors, except where noted in the tables. Median follow-up is 10 years.

2.6 Correlation of TS expression with patient and tumor characteristics

Four hundred eighty-eight samples were assessable for TS staining of invasive tumor. The level of TS expression according to patient and tumor characteristics was examined separately for the 210 node-negative and the 278 node-positive patients. Overall, 71% of the tumors in node-negative patients and 70% of those in node-positive patients were classified as high TS. Breakdowns according to patient and tumor characteristics showed little variation and there are no statistically significant relationships (2-sided p-values, results not shown). Thus, the TS staining intensity was not significantly associated with any of the following: Tumor size, ER status, PR status, histologic tumor grade, vessel invasion or histologic type, in both node-negative and node-positive tumors.

The TS staining in invasive breast tumors was predominantly a granular cytoplasmic staining pattern in tumor cells. TS staining was also present in lymphocytes adjacent to tumors and minor crossreactivity was observed with fibroblasts and other elements of connective tissue. Heterogeneity of TS staining was apparent both within individual tumors and between different tumors.

2.7 Correlation of TS Expression with Outcome and Treatment

Tables 1-4 show the correlations between TS expression, disease-free survival, overall survival and methods of treatment for both node-negative and node-positive patients. In these tables a “p-value” of 0.1 or less demonstrates that a significant correlation exists. P-values in excess of 0.1 suggest that no correlation can be drawn based on the data provided.

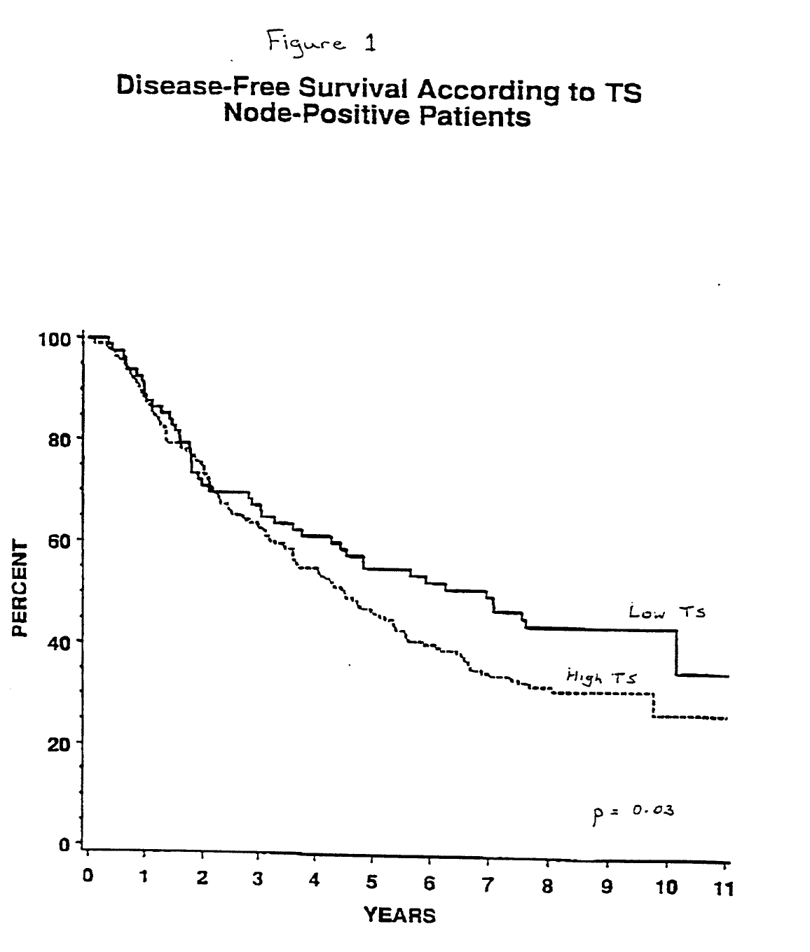

Table 1 shows Disease-Free Survival (DFS) according to TS level for both node-negative and node-positive patients. As can be seen from the data and p values, there is no significant difference between the low and high TS groups in node-negative patients (p=0.52). However, there is a DFS advantage for the low TS group of the node-positive patients (p=0.07 with no adjustments; p=0.03 after adjusting for treatment, Table 1 and FIG. 1).

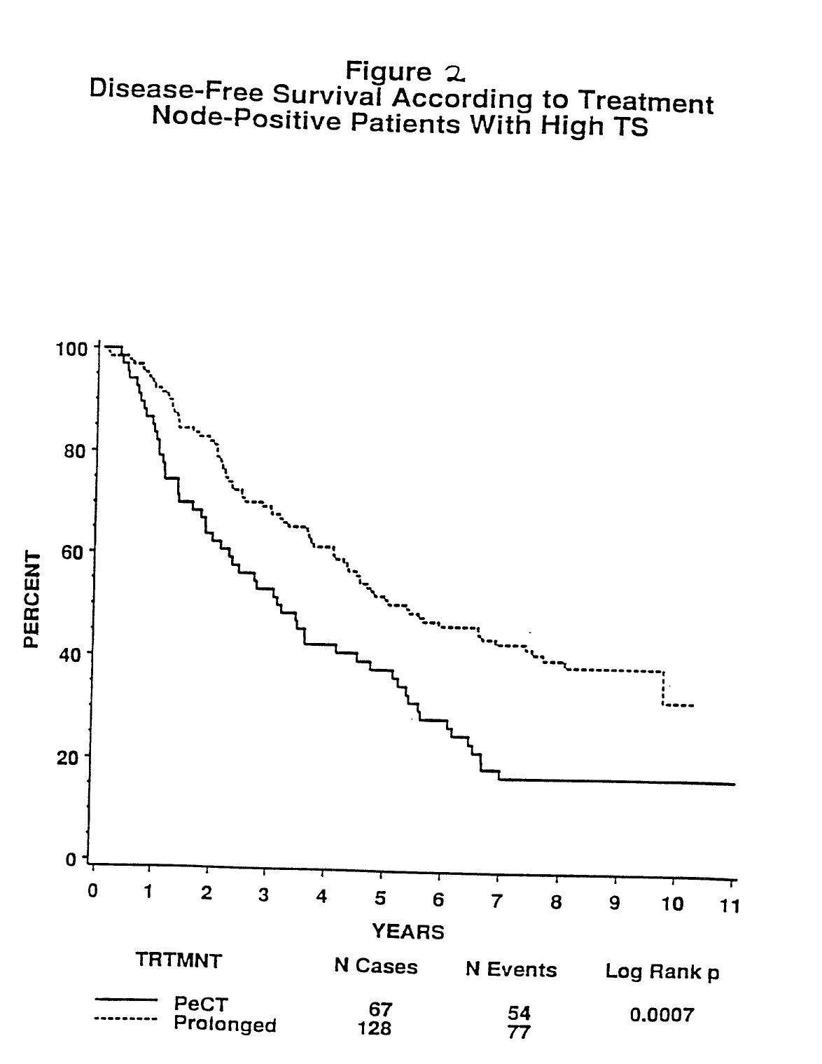

Table 2 shows DFS according to treatment assignment separately for the low TS and the high TS groups (also divided into node-positive and node-negative groups). In the node-positive group there is a disease-free advantage for patients receiving the prolonged treatment for both low TS and high TS patients, but the advantage is greater in the high TS group (see FIG. 2, p=0.001 in the high TS group; p=0.26 in the low TS group). However, the difference in treatment effectiveness between the low TS and the high TS group is not statistically significant (p=0.48 for the interaction between treatment group and TS in a Cox proportional hazards model).

Table 3 shows the breakdowns for overall survival (OS). The results are parallel to the results for DFS. Specifically, Table 3 shows OS according to TS level for the node-negative patients and node-positive patients. There is no significant difference between the low and high TS groups of node-negative patients (p=0.52). For the node-positive patients, there is a trend for an OS advantage for the low TS group (p=0.06, see FIG. 3). Table 4 shows OS according to treatment assignment separately for the low TS and the high TS groups of node-negative and node-positive patients. In node-negative patients, no correlation is seen. However, in node-positive patients there is a survival advantage in each group (high and low TS) for patients receiving the prolonged treatment, but the advantage is greater and significant only in the high TS group (see FIG. 4, p=0.01 in the high TS group; p=0.28 in the low TS group).

TABLE 1
Five and Ten-Year Disease-Free Survival (DFS)
According to Level of Thymidylate Expression,
Treatment and Nodal Status

| | | | 5-yr DFS | 10-yr DFS | |
|---|---|---|---|---|---|
| N cases | | N failures | (% ± SE) | (% ± SE) | P value |
| Node-Negative: | | | | | |
| TS low | 62 | 24 | 66 ± 6 | 61 ± 6 | 0.52 |
| TS high | 148 | 66 | 65 ± 4 | 50 ± 6 | |
| No CT: | | | | | |
| TS low | 18 | 6 | 67 ± 11 | 67 ± 11 | 0.36 |
| TS high | 53 | 24 | 60 ± 7 | 53 ± 7 | |
| PE CT: | | | | | |
| TS low | 44 | 18 | 66 ± 7 | 59 ± 8 | 0.90 |
| TS high | 95 | 42 | 68 ± 5 | 47 ± 8 | |
| Node-Positive: | | | | | |
| TS low | 83 | 46 | 55 ± 5 | 44 ± 6 | 0.03 |
| TS high | 195 | 131 | 46 ± 4 | 27 ± 5 | |
| PE CT: | | | | | |
| TS low | 36 | 22 | 53 ± 8 | 39 ± 9 | 0.05 |
| TS high | 67 | 54 | 38 ± 6 | 17 ± 5 | |
| Prolonged: | | | | | |
| TS low | 47 | 24 | 57 ± 7 | 48 ± 7 | 0.28 |
| TS high | 128 | 77 | 51 ± 4 | 33 ± 7 | |

TABLE 2
Five and Ten-Year Disease-Free Survival (DFS)
According to Treatment, Level of Thymidylate
Expression and Nodal Status

| | | | | |
|---|---|---|---|---|
| | | 5-yr DFS | 10-yr DFS | |
| N cases | N failures | (% ± SE) | (% ± SE) | P value |

Node-Negative:

| | | | | | |
|---|---|---|---|---|---|
| NoCT | 71 | 30 | 62 ± 6 | 56 ± 6 | 0.90 |
| PeCT | 139 | 60 | 67 ± 4 | 51 ± 6 | |

Thymidylate Synthase Low:

| | | | | | |
|---|---|---|---|---|---|
| NoCT | 18 | 6 | 67 ± 11 | 67 ± 11 | 0.53 |
| PeCT | 44 | 18 | 66 ± 7 | 59 ± 8 | |

Thymidylate Synthase High:

| | | | | | |
|---|---|---|---|---|---|
| NoCT | 53 | 24 | 60 ± 7 | 53 ± 7 | 0.78 |
| PeCT | 95 | 42 | 68 ± 5 | 47 ± 8 | |

Node-Positive:

| | | | | | |
|---|---|---|---|---|---|
| PeCT | 103 | 76 | 43 ± 5 | 25 ± 4 | 0.0007 |
| Prolonged | 175 | 101 | 53 ± 4 | 37 ± 5 | |

Thymidylate Synthase Low:

| | | | | | |
|---|---|---|---|---|---|
| PeCT | 36 | 22 | 53 ± 8 | 39 ± 9 | 0.15 |
| Prolonged | 47 | 24 | 57 ± 7 | 48 ± 7 | |

Thymidylate Synthase High:

| | | | | | |
|---|---|---|---|---|---|
| PeCT | 67 | 54 | 38 ± 6 | 17 ± 5 | 0.001 |
| Prolonged | 128 | 77 | 51 ± 4 | 33 ± 7 | |

TABLE 3
Five and Ten-Year Overall Survival (OS)
According to Level of Thymidylate Expression,
Treatment and Nodal Status

| | | | | |
|---|---|---|---|---|
| | | 5-yr OS | 10-yr OS | |
| N cases | N failures | (% ± SE) | (% ± SE) | P value |

Node-Negative:

| | | | | | |
|---|---|---|---|---|---|
| TS low | 62 | 16 | 81 ± 5 | 73 ± 6 | 0.52 |
| TS high | 148 | 44 | 80 ± 3 | 63 ± 6 | |

No CT:

| | | | | | |
|---|---|---|---|---|---|
| TS low | 18 | 3 | 83 ± 9 | 83 ± 9 | 0.18 |
| TS high | 53 | 18 | 77 ± 6 | 59 ± 10 | |

PE CT:

| | | | | | |
|---|---|---|---|---|---|
| TS low | 44 | 13 | 80 ± 6 | 70 ± 7 | 0.84 |
| TS high | 95 | 26 | 82 ± 4 | 65 ± 8 | |

Node-Positive:

| | | | | | |
|---|---|---|---|---|---|
| TS low | 83 | 34 | 73 ± 5 | 49 ± 8 | 0.06 |
| TS high | 195 | 102 | 64 ± 3 | 41 ± 5 | |

PE CT:

| | | | | | |
|---|---|---|---|---|---|
| TS low | 36 | 17 | 66 ± 8 | 40 ± 12 | 0.15 |
| TS high | 67 | 42 | 55 ± 6 | 33 ± 6 | |

Prolonged:

| | | | | | |
|---|---|---|---|---|---|
| TS low | 47 | 17 | 79 ± 6 | 56 ± 11 | 0.23 |
| TS high | 128 | 60 | 70 ± 4 | 45 ± 7 | |

TABLE 4
Five and Ten-Year Overall Survival (OS)
According to Treatment, Level of
Thymidylate Expression and Nodal Status

| | | | | |
|---|---|---|---|---|
| | | 5-yr OS | 10-yr OS | |
| N cases | N failures | (% ± SE) | (% ± SE) | P value |

Node-Negative:

| | | | | | |
|---|---|---|---|---|---|
| NoCT | 71 | 21 | 79 ± 5 | 66 ± 7 | 0.94 |
| PeCT | 139 | 39 | 81 ± 3 | 67 ± 6 | |

Thymidylate Synthase Low:

| | | | | | |
|---|---|---|---|---|---|
| NoCT | 18 | 3 | 83 ± 9 | 83 ± 9 | 0.30 |
| PeCT | 44 | 13 | 80 ± 6 | 70 ± 7 | |

Thymidylate Synthase High:

| | | | | | |
|---|---|---|---|---|---|
| NoCT | 53 | 18 | 77 ± 6 | 59 ± 10 | 0.46 |
| PeCT | 95 | 26 | 82 ± 4 | 65 ± 8 | |

Node-Positive:

| | | | | | |
|---|---|---|---|---|---|
| PeCT | 103 | 59 | 59 ± 5 | 33 ± 7 | 0.006 |
| Prolonged | 175 | 77 | 72 ± 3 | 48 ± 6 | |

Thymidylate Synthase Low:

| | | | | | |
|---|---|---|---|---|---|
| PeCT | 36 | 17 | 66 ± 8 | 40 ± 12 | 0.28 |
| Prolonged | 47 | 17 | 79 ± 6 | 56 ± 11 | |

Thymidylate Synthase High:

| | | | | | |
|---|---|---|---|---|---|
| PeCT | 67 | 42 | 55 ± 6 | 33 ± 6 | 0.01 |
| Prolonged | 128 | 60 | 70 ± 4 | 45 ± 7 | |

All publications, patents and patent applications mentioned in this specification are herein incorporated by reference into the specification to the same extent as if each individual publication, patent or patent application was specifically and individually indicated to be incorporated herein by reference.

Although the foregoing invention has been described in some detail by way of illustration and example for purposes of clarity of understanding, it will be obvious that certain changes and modifications may be practiced within the scope of the appended claims.

What is claimed is:

1. A method for determining the prognosis of a patient afflicted with breast cancer, comprising;
(a) obtaining a solid breast tumor tissue sample from said patient;
(b) measuring the level of thymidylate synthase expression in said tissue sample; and
(c) comparing said thymidylate synthase expression level with a thymidylate synthase expression levels from a group of standard breast cancer tissue samples, said standards having known thymidylate synthase expression levels, known disease-free survival rates and known overall survival rates, to determine the prognosis of said patient.

2. A method in accordance with claim 1, wherein said measuring comprises contacting said tissue sample with an antibody specific for thymidylate synthase.

3. A method in accordance with claim 1, wherein said patient is node-positive.

4. A method in accordance with claim 1, wherein said measuring uses an avidin-biotin complex immunohistochemical technique.

5. A method in accordance with claim 2, wherein said antibody is a TS 106 monoclonal antibody.

6. A method for predicting the benefit of chemotherapy for a patient afflicted with breast cancer, comprising;
(a) obtaining a solid breast tumor tissue sample from said patient;
(b) measuring the level of thymidylate synthase expression in said tissue sample; and
(c) comparing said thymidylate synthase expression level with thymidylate synthase expression levels from a group of standard breast cancer tissue samples, said standards having known thymidylate synthase expression levels, known courses of chemotherapeutic treatment, known disease-free survival rates and known overall survival rates, to predict the benefit of chemotherapy for said patient.

7. A method in accordance with claim 6, wherein said measuring comprises contacting said tissue sample with an antibody specific for thymidylate synthase.

8. A method in accordance with claim 6, wherein said patient is node-positive.

9. A method in accordance with claim 6, wherein said measuring uses an avidin-biotin complex immunohistochemical technique.

10. A method in accordance with claim 7, wherein said antibody is a TS 106 monoclonal antibody.
